(19) United States
(12) Patent Application Publication
(10) Pub. No.: US 2004/0073306 A1
(43) Pub. Date: Apr. 15, 2004

(54) Implant

(75) Inventors: Juergen Eichhorn, Mittelfels/Scheibelsgrub (DE); Nicola Giordano, Villingen-Schwenningen (DE); Karl-Ernst Kienzle, Tuttlingen-Moehringen (DE); Tom Schweitzer, Tuttlingen (DE)

Correspondence Address: LAW OFFICE OF BARRY R LIPSITZ, 755 MAIN STREET, MONROE, CT, 06468, US

(73) Assignee: AESCULAP AG & Co. KG, Tuttlingen (DE)

(21) Appl. No.: 10/631,466

(22) Filed: Jul. 30, 2003

Related U.S. Application Data
(63) Continuation of application No. PCT/EP02/01094, filed on Feb. 2, 2002.

(30) Foreign Application Priority Data
Feb. 2, 2001 (DE) 201 01 791.1

(51) Int. Cl. A61F 2/08

(52) U.S. Cl. 623/13.11

(57) ABSTRACT

In order to facilitate insertion and reduce movement of the tendon replacement part in the channel in an implant for fastening of a tendon replacement part to a channel receiving the tendon replacement part in the region of the tibia and/or femur close to the knee, with an abutment element abutting against the channel and with a connecting element, wherein the tendon replacement part and the abutment element may be coupled by means of the connecting element, it is proposed that the abutment element and the connecting element defining a longitudinal direction are configured and reconciled with one another such that the tendon replacement part is fixable in the channel by relative movement between the abutment element and the connecting element in longitudinal direction.

The present disclosure relates to the subject matter disclosed in international application PCT/EP 02/01094 of Feb. 2, 2002, which is incorporated herein by reference in its entity and for all purposes.

### BACKGROUND OF THE INVENTION

The invention relates to an implant for fastening of a tendon replacement part to a channel receiving the tendon replacement part in the region of the tibia and/or femur close to the knee, with an abutment element abutting against the channel and with a connecting element, wherein the tendon replacement part and the abutment element may be coupled by means of the connecting element.

Such implants are used in the reconstruction of cruciate ligaments, for example. In this case, hamstring tendons (semitendinosus, gracilis or plantaris tendon) or BTB transplants (patellar tendon) are used as tendon replacement part. A polyester tape or polyester thread is usually used for connecting elements, with which the tendon replacement part is anchored in the channel on an extra-ossary titanium anchoring plate remote from the joint.

On use of the previously usual connecting elements, the so-called “bungee effect” occurs, which results from the elasticity of the connecting elements used. An undesirable consequence of the bungee effect is a lower primary stability after the implant is inserted and also a slight, but nevertheless possible, longitudinal movement (micro-movement) of the transplant in the channel. The latter interferes with ingrowth of the transplant in the channel and therefore renders very careful aftercare necessary. In addition, as a result of this longitudinal movement in the joint a trumpet-shaped broadening of the channels close to the joint can occur, which is referred to as a “bone tunnel enlargement”.

Moreover, it is very difficult for an operating surgeon to fix the tendon replacement part in the channel without play, since generally the polyester thread must be knotted on the titanium anchoring plate.

### SUMMARY OF THE INVENTION

The present invention improves an implant of the above-described type in that the abutment element and the connecting element defining a longitudinal direction are configured and reconciled with one another such that the tendon replacement part may is tensingly fixable in the channel by relative movement between the abutment element and the connecting element in longitudinal direction.

Such a configuration of the implant enables the tendon replacement part to be fixed in the channel only through relative movement between the abutment element and the connecting element. Complicated knotting, e.g. of polyester threads serving as connecting elements on a titanium anchoring plate serving as abutment element, is unnecessary. The tendon replacement part is clamped by only moving the connecting element relative to the abutment element, in which case the position respectively assumed between the connecting element and the abutment element as a result of the relative movement is unchangeable. Thus, the present invention leads to an improvement of the implant such that it can be inserted more simply and the “bungee effect” and the “bone tunnel enlargement” are prevented or at least diminished.

To improve the healing process and avoid reactions of rejection, it is advantageous if the connecting element and/or the abutment element are made from a material body compatible material.

It is favourable if the body compatible material is a resorbable plastic. This facilitates in particular a renewed surgical procedure, since the resorbable material can be drilled without any problem. In particular, it cannot wrap around the drill during drilling, as is possible with a polyester tape in the above-described anchoring methods. In addition, it can grow in completely and be absorbed without any problem at all as a result of low accumulations of material.

In principle, it can be provided that the abutment element has at least one passage recess for the connecting element, so that the abutment element and the connecting element are movable relative to one another transversely to the longitudinal direction. Such a configuration enables the abutment element and the connecting element to be fixed relative to one another by an additional movement transversely to the longitudinal direction of the connecting element. Fixing can be reinforced with a movement transversely to the longitudinal direction in addition to a movement in the longitudinal direction.

In a further preferred embodiment of the invention it can be provided that the abutment element has at least one passage recess for the connecting element, so that the connecting element and the abutment element are displaceable relative to one another, and that the abutment element has at least one fixture recess, wherein the at least one passage recess and the at least one fixture recess are connected to one another. Thus, as a result of displacement of the connecting element relative to the abutment element, the connecting element is able to extend at least partially into the fixture recess of the abutment element and both elements can be fixed relative to one another.

It is advantageous in this case if the connecting element comprises a plurality of fixture recesses, which are arranged in the longitudinal direction of the connecting element. The stability of the connection of both elements is additionally increased as a result of this. Moreover, a plurality of fixing positions may thus be provided between the abutment element and the connecting element.

It is particularly favourable if adjacent fixture recesses are spaced equidistant from one another. This allows fixing positions to be established at defined intervals between the connecting element and the abutment element.

It can be advantageous if adjacent fixture recesses are separated from one another. This enables each fixture recess to be selectively brought into engagement with the abutment element.

In a further preferred embodiment of the invention it can be provided that the connecting element is provided with an engagement strip formed by fixture recesses. This engagement strip enables any desired positions to be provided between the abutment element and connecting element, which reduces the preparation effort overall, since no polyester tape needs to be knotted to a defined length. This reduces the potential for error during the surgical procedure. Moreover, it also saves time, since the knot of a polyester tape does not have to be tightened, as there is no knot present.

It is favourable in this case if the connecting element has at least two engagement strips. The stability of the connection is additionally increased as a result of this.

It can be advantageously provided that a side face of the connecting element is provided with an engagement strip. The engagement strip can be attached particularly simply on one side face of the connecting element.

It can be additionally advantageous if an engagement strip is arranged on the connecting element between side faces of the connecting element. This allows the connecting element to slide along the side faces practically free from friction.

It is particularly favourable if the engagement strip is configured in a sawtooth shape. This enables unidirectional displacement of the connecting element relative to the abutment element. Movement in the opposite direction is then no longer possible, the abutment element is fixed in this direction relative to the connecting element. In this way, the tendon replacement part can be clamped simply in the channel. In particular, such a connecting element has a lower elasticity than a polyester tape, so that practically no bungee effect occurs, while all the advantages of a fixture of the implant remote from the joint are retained.

In a further preferred embodiment it can be provided that an engagement strip comprises rectangular recesses as fixture recesses. Such recesses can be produced particularly simply and with corresponding elements immersing into them allow a fixture that is practically free from play.

It can also be advantageous if the abutment element has a holding member, which is immersible into a fixture recess and by means of which the abutment element may be connected to the connecting element. The connection is enabled in a simple manner by the holding member immersing into the fixture recess. This can occur as a result of the relative movement of connection element and abutment element in the longitudinal direction of the connecting element, for example, by holding members immersing into fixture recesses of an engagement strip configured in a sawtooth shape.

To increase the stability of the connection it can be provided that a holding member comprises a plurality of holding teeth for simultaneous engagement into a plurality of fixture recesses.

It can be advantageously provided that a holding member is arranged at an angle to an abutment face of the abutment element. In this way, movement can be made easier in the longitudinal direction of the connecting element, but made difficult in the opposite direction, as a result of which the connection between the connecting element and abutment element is additionally strengthened.

In principle it can be provided that the abutment element is connectable to the connecting element as a result of a relative movement of the entire abutment element thereto. This configuration allows abutting the abutment element on the channel of the bone and moving the connecting element relative to the abutment element, e.g. drawing along this or passing it through it, in which case it is preferably only possible to draw the connecting element out of the channel and in this way clamp the tendon replacement part, whereas a movement between the abutment element and connecting element in the opposite direction is prevented because of the respective configuration.

It is particularly favourable if the connecting element is a body that is inflexible in the longitudinal direction. A bungee effect is effectively prevented because of this lower elasticity in comparison to anchorage of the tendon replacement part by using polyester tapes. This increases the primary stability directly after the surgical procedure and additionally allows a more vigorous aftercare.

In a further preferred embodiment of the invention, it can be provided that the connecting element is dimensioned such that it has a larger width in a transverse direction to its longitudinal direction than in a transverse direction perpendicular thereto. Such connecting elements can be produced particularly simply and as the case may be provided along guide recesses of side faces.

It is particularly advantageous if the connecting element has an essentially rectangular cross-section at least in the region extending in the drilling channel. A rectangular cross-section is particularly simple to produce. Moreover, one or more side faces can be easily provided with guide recesses.

However, in a further preferred embodiment of the invention it can be provided that the connecting element is configured to be essentially rotationally symmetric in its longitudinal direction. This has the advantage, particularly in the case of a channel formed by drilling, that the connecting element can fill the drilling channel to an optimum degree. Moreover, it is particularly simple to produce.

In this case it can be favourable if in the case of an essentially rotationally symmetric configuration of the connecting element, a fixture recess extends around the periphery of the connecting element. This allows a secure connection with the abutment element to be achieved, irrespective of the orientation of the connecting element inside the channel.

It can be advantageous if adjacent fixture recesses are separated by a spherical element. This increases in particular the stability of the connecting element and reduces its elasticity.

In an alternative, likewise advantageous embodiment, it can be provided that the holding part of the abutment element engages in the longitudinal direction into a fixture recess. This facilitates fixture solely on the basis of a relative movement between the connecting element and the abutment element in the longitudinal direction of the connecting element.

In a further preferred embodiment of the invention, it can be additionally provided that the abutment element may be connected to the connecting element by rotation relative thereto. This enables, for example, an unhindered movement in the longitudinal direction of the connecting element, while a rotation ultimately leads to a connection of both elements.

It can be advantageous in this case if the abutment element has a holding member, which is engagable into a fixture recess of the connecting element by rotation of the abutment element relative to the connecting element. In this way, it is possible to move both elements practically free from friction in the longitudinal direction of the connecting element, whereas the rotation leads to a connection of the two elements. A further advantage of this connection is that it can be easily released by again twisting to allow, for example, the tendon replacement part to be re-tensioned or relieved of tension.

In principle, it can be advantageous if the connecting element is flexible transversely to its longitudinal direction. This allows a simple connection between the connecting element and the abutment element and simplifies insertion of the implant.

In principle, the abutment element can be capable of being fixed positively to the connecting element. However, it is particularly advantageous if the abutment element is fixable non-positively to the connecting element. This additionally increases the stability of the connection of both elements.

In a further advantageous embodiment of the invention, it can be provided that the relative movement between the abutment element and the connecting element for fixing the abutment element to the connecting element is performed from a non-fixed position into a fixing position. As a result, it is possible to move the connecting element relative to the abutment element firstly without application of a high force, whereas fixture occurs in a fixing position that is only assumed as a result of the relative movement.

Advantageously, it can be provided that the end of the connecting element facing the tendon replacement part has a tendon replacement part receptacle for passage or suspension of the tendon replacement part. This enables the tendon replacement part to be connected to the connecting element particularly easily. In principle, other types of connection, e.g. by suturing or gluing, would also be conceivable.

In this case, it can be favourable if the tendon replacement part receptacle is configured in a loop shape. This has a particularly high stability overall. Moreover, the tendon replacement part can be easily passed through such a loop-shaped tendon replacement part receptacle.

In principle, the shape of the loop-shaped tendon replacement part receptacle can be as desired. However, it is advantageous if it has an essentially round loop shape. Any sharp edges or pointed corners, which could cause damage to the tendon replacement part, can be avoided as a result of this.

However, it is also favourable if the loop-shaped tendon replacement part receptacle is essentially triangular. This permits a leg of the tendon replacement part receptacle extending essentially transversely to the longitudinal direction of the connecting element, against which the tendon replacement part can be laid. This causes an optimum transfer of force between the tendon replacement part and the connecting element.

Moreover, it can be advantageous if the tendon replacement part receptacle is configured in a hook shape. In particular in the case of BTA transplants, in which a free end is provided with a rectangular plate having two drilled holes, these can be connected to the connecting elements in a particularly simple manner, since the hook-shaped tendon replacement part receptacle can be directed through both holes.

In this case, it can be particularly favourable if a locking element is provided on the free end of the hook-shaped tendon replacement part receptacle and at least one locking element receptacle is provided on the connecting element to move the hook-shaped tendon replacement part receptacle into a loop-shaped tendon replacement part receptacle and lock it in position. This has the advantage that the tendon replacement part located in the tendon replacement part receptacle can be secured against sliding out unintentionally. In addition, the end of the connecting element forming the tendon replacement part receptacle can be sufficiently elastic to facilitate locking. Preparation of the implant is simplified in this way.

In principle, any desired cross-sections of the tendon replacement part receptacle would be conceivable. However, it is of advantage if these are round, oval or rectangular, at least in sections. Round and/or oval cross-sections have the advantage that damage to the tendon implant can be prevented. Rectangular cross-sections can be produced particularly simply and have an increased stability.

It is particularly advantageous if the abutment element can be screwed to the connecting element. This allows the position of the connecting element in relation to the abutment element to be infinitely controlled. As a result, the tendon replacement part can be tensioned in an optimum manner as desired.

It can be favourable in this case if the passage recess of the abutment element is provided with an internal thread, and if the connecting element is provided with a corresponding external thread. Both threads may be produced particularly simply on the two elements.

Advantageously it can be additionally provided that the abutment element has a section extending into the channel. In contrast to the previously used anchoring plate, which only abuts on the outside, a cavity in the bone can be avoided as a result of this, thus enabling better healing in particular.

It is favourable in this case if the extending section is configured to be rotationally symmetric. In this way, it can be optimally adapted to a drilled channel, as a result of which the play between the abutment element and the bone is minimised.

It would be conceivable that the extending section has a constant cross-section in the longitudinal direction. However, it is particularly advantageous if the extending section has a cross-section which decreases in the longitudinal direction of the passage recess towards the tendon replacement part. Such a, for example, conical configuration allows the abutment element to be simply clamped in the channel at its extending section.

It can also be advantageous if the abutment element has an abutment region abutting externally against the channel and at least partially covering the channel. Such an abutment region prevents the abutment element from immersing into the channel. With the abutment region a stop for the abutment element on the bone is practically formed in order to prevent the abutment element from penetrating into the channel.

In this case, it can be provided that the abutment region has an external face directed away from the drilling channel, which runs essentially in a plane transversely to the longitudinal direction of the passage recess. Such an abutment element can be produced in a particularly simple manner.

However, it can be advantageous if the abutment region has an external face directed away from the drilling channel, which with a plane transversely to the longitudinal direction of the passage recess encloses an angle, which corresponds to an angle formed between the channel and a passage face formed by the channel in the tibia or the femur. Particularly with a drilled hole obliquely entering the bone from the bone surface, such a configuration of the abutment region enables the abutment element to abut against the bone in an optimum manner. In particular, the abutment element only projects minimally from the bone in this way.

It is favourable if the abutment region has a larger cross-section than the immersing section. In this way, it is possible to cover the channel completely and to form a stop on the bone.

In addition, it can be advantageously provided that the abutment region is pivotally disposed on the immersing section. This has the advantage that the abutment region is always oriented parallel to the surface of the bone and thus only projects minimally from the bone. Moreover, in this way the force acting on the bone by tensioning the tendon replacement part via the abutment element can be transferred in an optimum manner onto the entire edge of the channel on the bone surface.

In a preferred embodiment of the invention it can be provided that the holding member is displaceably disposed on the abutment element in a direction transverse to the passage recess. In this way, the holding member can be moved purposefully from a release position into a locking position and vice versa.

To assure a defined movement of the holding member, it is advantageous if a guide recess running transversely to the passage recess is provided for guiding and supporting the holding member.

In addition, it is favourable if the holding member is biased towards the passage recess. As a result, the holding member can be held constantly in a locking position and secured as a result of the prestress.

A particularly simple structure of the implant results if an elastic element is provided to generate a prestress.

The elastic element is advantageously configured in one piece with the holding member. A restraining element in one piece comprising both the holding member and the elastic element reduces the number of structural elements necessary. Moreover, the assembly of the abutment element is substantially simplified.

According to a preferred embodiment of the invention it can be provided that the fixture recess comprises a fixture recess face oriented transversely, and one oriented obliquely, to the longitudinal direction of the connecting element. This enables the abutment element to move in only one direction relative to the connecting element. The fixture recess face oriented obliquely to the longitudinal direction in this case serves as a slide face, the fixture recess face oriented transversely to the longitudinal direction of the connecting element serving as a stop for the abutment element or parts thereof.

### BRIEF DESCRIPTION OF THE DRAWINGS

The following description serves to provide a more detailed explanation in conjunction with the drawing:

FIG. 1 is a schematic view of a knee joint with a tendon replacement part fixed in the femur and in the tibia;

FIG. 2 is a cross-sectional view of a first embodiment of an abutment element according to the invention;

FIG. 3 is a top view onto the abutment element from FIG. 2;

FIG. 4 is a cross-sectional view of a second embodiment of an abutment element;

FIG. 5 is a cross-sectional view of a third embodiment of an abutment element;

FIG. 6 is a cross-sectional view of a fourth embodiment of an abutment element;

FIG. 7 is a schematic side view of a loop-shaped tendon replacement part receptacle;

FIG. 8 is a side view in the direction of arrow A in FIG. 7;

FIG. 9 is a cross-sectional view along line 9-9 in FIG. 7 or FIG. 13;

FIG. 10 is a cross-sectional view along line 10-10 in FIG. 7 or FIG. 13;

FIG. 11 is a cross-sectional view along line 11-11 in FIG. 7 or FIG. 13;

FIG. 12 is a cross-sectional view along line 12-12 in FIG. 7 or FIG. 13;

FIG. 13 is a side view of a loop-shaped tendon replacement part receptacle in the shape of a triangle;

FIG. 14 is a side view of a lockable hook-shaped tendon replacement part receptacle;

FIG. 15 is a side view in the direction of arrow B in FIG. 14;

FIG. 16 is a side view of a first embodiment of a connecting element with associated abutment element;

FIG. 17 is a cross-sectional view along line 17-17 in FIG. 16;

FIG. 18 is a side view of a second embodiment of a connecting element with associated abutment element;

FIG. 19 is a top view in the direction of arrow C in FIG. 18;

FIG. 20 is a side view in the direction of arrow D in FIG. 18;

FIG. 21 is a cross-sectional view through a third embodiment of a connecting element with associated abutment element;

FIG. 22 is a top view onto the abutment element in FIG. 21 in the direction of arrow E;

FIG. 23 is a side view of the connecting element from FIG. 21 in the direction of arrow F;

FIG. 24 is a side view of a fourth embodiment of a connecting element with associated abutment element;

FIG. 25 is a top view onto the abutment element in FIG. 24 in the direction of arrow G;

FIG. 26 is a side view of the connecting element from FIG. 24 in the direction of arrow H;

FIG. 27 is a side view of a fifth embodiment of a connecting element with associated abutment element;

FIG. 28 is a top view onto the abutment element from FIG. 27 in the direction of arrow I;

FIG. 29 is a side view of a sixth embodiment of a connecting element with associated abutment element;

FIG. 30 is a top view onto the abutment element from FIG. 29 in the direction of arrow K;

FIG. 31 is a side view of the connecting element from FIG. 29 in the direction of arrow L;

FIG. 32 is a side view of a seventh embodiment of a connecting element with associated abutment element;

FIG. 33 is a top view onto the abutment element from FIG. 32 in the direction of arrow M;

FIG. 34 is a side view of an eighth embodiment of a connecting element with associated abutment element;

FIG. 35 is a top view onto the abutment element from FIG. 34 in the direction of arrow N;

FIG. 36 is a view in partial longitudinal section through a ninth embodiment of a connecting element with associated abutment element;

FIG. 37 is a side view of the ninth embodiment shown in partial section in FIG. 36, and

FIG. 38 is a top view onto a restraining element forming a component of the ninth embodiment.

### DESCRIPTION OF THE PREFERRED EMBODIMENTS

In order to replace a destroyed cruciate ligament in a knee joint 100, longitudinal channels 106 and 108, which are essentially aligned with one another when the knee joint 100 is straightened and extend from the front to the rear of the knee joint 100 to pass obliquely through this, are respectively drilled into the head 102 of the femur and the head 104 of the tibia.

A cruciate ligament replacement part 116, which comprises a band-shaped piece 118 of a tendon replacement part, e.g. a part of the patellar tendon, at the ends of which loop-shaped ends 124 and 126, drawn through openings 120, 122, of fastening rails 128, 130 preferably made from resorbable plastic are arranged, is drawn into channel sections 112 or 114 of the longitudinal channels 106 or 108. These channel sections are widened in diameter in one step and arranged to face the joint cavity 110 located between the head 104 of the tibia and the head 102 of the femur. The elongated fastening rails 128 or 130, which respectively pass completely through the longitudinal channels 106 or 108 and project out of the head 102 of the femur or head 104 of the tibia, have a respective coupling section 136 or 138, which faces their respective second free end 132 or 134 and can be respectively connected to a corresponding region of a fastening block 140 or 142, which partially extends into the longitudinal channels 106 or 108 and partially abuts against the outside of the head 102 of the femur or the head 104 of the tibia.

During insertion of the cruciate ligament replacement part 116, the band-shaped piece 118 is drawn in as far as the stop of the channel sections 112 or 114 formed by the single-step widened portion and prestressed by the fastening rails 128 and 130 being drawn in the opposite direction out of the knee joint 100 and moved relative to the fastening blocks 140 and 142, that is until the band-shaped piece 118 is positioned inside the channel sections 112 and 114 in the desired manner.

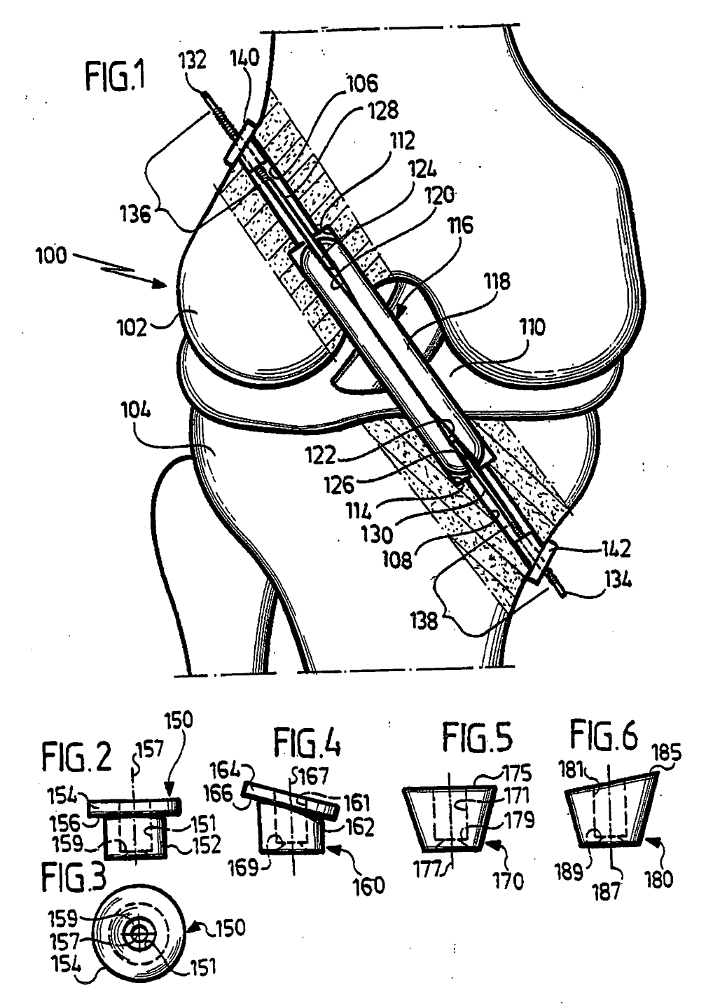

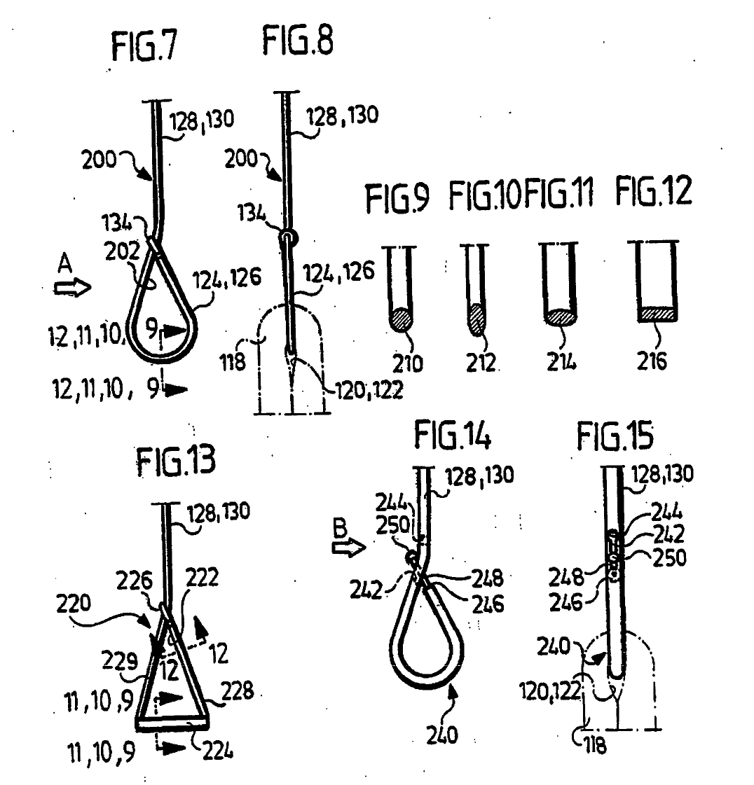

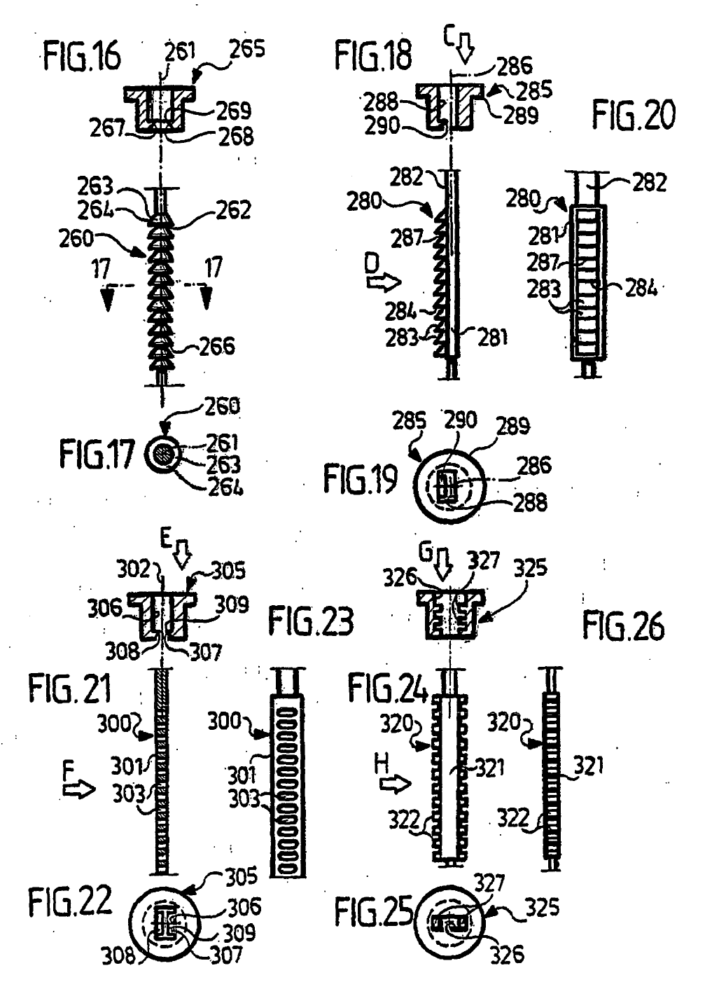

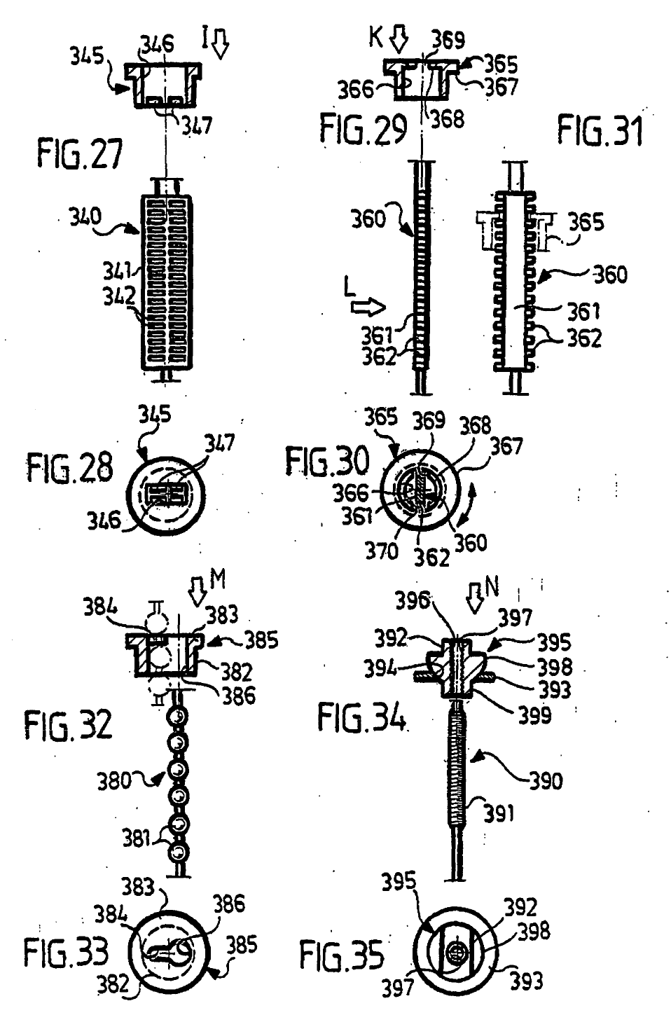

The fastening blocks 140 and 142 and also the fastening rails 128 and 130 with the loop-shaped ends 124 and 126 arranged thereon can be structured differently. Various configurations of the individual elements are shown in FIGS. 2 to 35 and explained in more detail below. In this case, the base forms of the fastening blocks 140, 142 can be combined as desired with loop-shaped ends 124, 126 and also with different coupling sections 136, 138 with respectively corresponding configurations of fastening blocks 140, 142. Therefore, the individual elements of the cruciate ligament replacement part 116 are each explained separately below.

Four different basic types of fastening blocks 140, 142 are shown in FIGS. 2 to 6. FIGS. 2 and 3 show a block 150 that is fully rotationally symmetric relative to the axis of symmetry 157 with a hollow cylindrical body section 152, which may be inserted into the longitudinal channel 106 or 108, and with a head 154 of larger diameter adjoining it. The side face 156 of the head 154 facing the body section 152 serves as a stop on the head 102 of the femur or head 104 of the tibia for the block 150 in order to prevent penetration into the longitudinal channel 106 or 108.

Contrary to the case with block 150, with block 160 shown in FIG. 4, the head 164 projecting above the hollow cylindrical body section 162 in diameter is inclined relative to the axis of symmetry 167 of the body section 162. As shown in FIG. 1, this provides the possibility of the body section 162 extending into the longitudinal channels 106 or 108 with its axis of symmetry 167 parallel to the longitudinal axis thereof, whereas the side face 166 of the head 164 abuts fully against the external face of the head 102 of the femur or the head 104 of the tibia in spite of the obliquely drilled longitudinal channels 106 or 108. In this way, the force acting on the side face 166 can be transferred onto the head 102 of the femur or the head 104 of the tibia in an optimum manner during tensioning of the cruciate ligament replacement part 116.

FIGS. 5 and 6 show blocks 170 and 180 that are conical with respect to their axes of symmetry 177 and 187 and respectively have a central hole 171 or 181. At their end with the largest diameter, they have a plane, ring-shaped external face 175 or 185, wherein external face 175 is oriented perpendicular to the axis of symmetry 177, whereas the external face 185 is inclined relative to the axis of symmetry 187. Both block 170 and block 180 are inserted into the longitudinal channels 106 or 108 from the outside and are clamped in these as a result of their conical shape. The inclined external face 185 is advantageous, in particular in the case of longitudinal channels 106 or 108 arranged obliquely in the head 102 of the femur or head 104 of the tibia, so that block 180 projects only minimally from the surface of the two bones.

At their end plunging into the longitudinal channel 106 or 108, blocks 150, 160, 170 and 180 have holding elements 159, 169, 179 and 189 projecting inwards towards the axes of symmetry 157, 167, 177 and 187 from the inside surface of the drilled holes 151, 161, 171 and 181. These are only shown schematically in FIGS. 2 to 6, possible alternative embodiments are described in more detail in association with FIGS. 16 to 35.

The loop-shaped ends 124 and 126 of the fastening rails 128 and 130 can be configured differently, as already indicated. Examples thereof are shown in FIGS. 7 to 15.

FIGS. 7 and 8 show a first embodiment of a loop 200, which defines an essentially drop-shaped passage 202, wherein a free end 134 of one of the fastening rails 128 or 130 is directed back on itself and is, for example, welded, glued, knotted or clamped thereto. A band-shaped piece 118 of a cruciate ligament replacement part 116 can then be passed through the passage 202, as is shown in FIG. 8.

FIGS. 9 to 12 show different possible cross-sections of the loop 200. FIG. 9 shows a circular cross-sectional area 210, FIG. 10 an elongated oval cross-sectional area 212, FIG. 11 shows an in transverse direction elongated oval cross-sectional area 214 and finally FIG. 12 shows a rectangular cross-sectional area 216. Each of the cross-sectional areas 210, 212, 214 and 216 shown in FIGS. 9 to 12 may be combined in any desired way with the shape of the loop 200 shown in FIGS. 7 and 8.

FIG. 13 shows a loop 220 slightly modified from loop 200, which defines a triangular passage 222 with the shape of an isosceles triangle, the base of which defines a holding section 224 running perpendicular to the longitudinal direction of the fastening rail 128 or 130. The two loop sections 228 and 229 of equal length are joined together at a connection point 226, which can be formed, for example, by a gluing point, a welding point or by clamping.

The holding section 224 can have different shapes in cross-section, e.g. circular or elongated oval cross-sectional areas 210, 212 or 214 shown in FIGS. 9 to 11 are conceivable.

In order to stabilise the shape of the loop 220, the cross-section of the two loop sections 228 and 229 of equal length is rectangular, as shown in FIG. 12. Like in the loop 200, the band-shaped piece 118 in loop 220 is directed through the passage 222 and abuts against the holding section 224.

A third embodiment of a loop 240 according to the invention is shown in FIGS. 14 and 15. The shape of the loop 240 essentially corresponds to that of loop 220. The essential difference is, however, that the loop 240 can be opened. For this, the fastening rail 128 or 130 has an elongated hole 242 extending in the longitudinal direction, at the end of which remote from the loop 240 an insertion opening 244 configured with a larger diameter than the elongated hole is arranged. The free end 246 of the fastening rail 148 or 130 is provided with a locking pin 248 projecting rectilinearly from the free end 246 in extension of the fastening rail 128, 130, said locking pin has a head 250.

For fastening the band-shaped piece 118 to the fastening rail 128, 130, the opened loop 240, which is hook-shaped in this position, is directed through one of the openings 120, 122 of the band-shaped piece 118. The locking pin 248 is passed completely through the insertion opening 244 with its head 250 and can then be displaced from the insertion opening 244 into the elongated hole 242. As a result, the band-shaped piece 118 is secured in the loop 240 forming a drop-shaped loop like loop 200 in a closed position.

FIGS. 16 to 35 show different variants of coupling sections 136 or 138 and corresponding holding elements 159, 169, 179 and 189 schematically shown in association with FIGS. 2 to 6. In association with FIGS. 16 to 33, fastening blocks are respectively used, which correspond in external structure to the block 150, i.e. are configured to be completely rotationally symmetric and have a hollow cylindrical body section. However, fastening blocks with a base body such as the blocks 160, 170 or 180 shown in FIGS. 4, 5 and 6 have are also possible.

FIG. 16 shows a section from a fastening rail 128 with a coupling section 260, which is formed completely rotationally symmetric to the longitudinal axis 261. The coupling section 260 is formed by a plurality of identical annular recesses 262, so that an elongated structure remains, which consists of the same number of cone bodies 263 having a spherical base area 266 oriented transversely to the longitudinal axis, wherein the point of each cone body 263 penetrates into the base area of the adjacent cone body. The coupling section 260 thus has a Christmas tree-like appearance in side view.

A block 265 serving as abutment element has a ring-shaped projection 267 corresponding to the annular recesses 262, which projects towards the longitudinal axis 261 and, like the cone bodies 263, has a sloping external face 268.

When connecting the block 265 to the fastening rail, the block 265 is pushed over the fastening rail until the external faces 268 slide over sloping cone faces 264 of the cone bodies 263. A connection is created between the block 265 and the fastening rail in the region of the coupling section 260 as soon as the catch face 269 of the projection 267 arranged to run transversely to the longitudinal axis 261 engages behind the cone base area 266. While it is still possible to slide the block 265 up further onto the fastening rail, it is not possible to pull it back because of the catch connection formed. To tension the band-shaped piece 118, one of the two fastening rails can be respectively displaced in stages in the longitudinal direction, that is precisely at the spacing between two cone base areas 266.

FIGS. 18 to 20 show an alternative configuration of a coupling section 280 with associated block 285. On a base body 281 of the coupling section 280 with a rectangular cross-section, a plurality of sawteeth 283 are spaced at equal distance on one side face 282, thus forming a toothed strip. Each sawtooth 283 has a tooth face 284 oriented transversely to the longitudinal axis 286. Along its longitudinal axis 286 the block 285 has a perforation 288 with a rectangular cross-section, wherein the cross-section of the perforation corresponds to the cross-section of the coupling section 280 in the region of the tooth faces 284. On their side remote from a head 289 of the block 285, the block 285 has a locking tooth 290, which can fill a fixture recess 287 formed between two sawteeth 283.

The block 285 can be locked in one direction with the fastening rail in the region of the coupling section 280, i.e. by a sloping side face of the locking tooth 290 sliding on over a sloping side face of one of the sawteeth 283 until the locking tooth 290 extends into the fixture recess 287. In this configuration of the coupling section 280 a stepwise adjustment of the prestress of the band-shaped piece 118 is possible.

A further coupling section 300 is shown in FIGS. 21 to 23. A base body 301 of the coupling section 300 with a rectangular cross-section is provided transversely to its longitudinal direction with essentially rectangular perforations 303, which are arranged equidistant along the base body 301. A corresponding block 305 serving as abutment element is provided with a perforation 306 likewise with a rectangular cross-section, wherein at one end this has two opposing locking teeth 307 and 308 respectively pointing from an inside wall of the perforation 306 towards the opposite wall.

For clamping the band-shaped piece 118 in position, the fastening rail is pushed through the perforation 306 of the block 305, in fact until the locking teeth 307 and 308 plunge into one of the perforations 303. Since each of the locking teeth 307 and 308 has a tooth face 309 oriented transversely to the longitudinal direction 302, movement of the block 305 relative to the fastening rail is only possible in one direction. By advancing the fastening rail further into the block 305, the band-shaped piece 118 can be tensioned in the desired manner in defined steps respectively corresponding to the distance between two perforations 303.

A further possibility of connecting a block 325 to a fastening rail is shown in FIGS. 24 to 26. A coupling section 320 comprises as base body 321 with a rectangular cross-section, wherein its narrow sides respectively have a row of rectangular teeth 322 spaced at equal distance. The block 325 has a perforation 326 of a loop-shaped base body likewise with a rectangular cross-section, wherein from the narrow side faces thereof a parallelepipedal tab with three locking teeth 327 arranged thereon respectively projects in the longitudinal direction of the coupling section 320, in fact such that they can extend into depressions formed between the teeth 322, when the block 325 is pushed over the coupling section 320.

A further conceivable coupling section 340 is shown in FIGS. 27 and 28. The principle corresponds to that in association with the coupling section 300 shown in FIGS. 21 to 23. However, two parallel rows of perforations 342 spaced at equal distance and running transversely to the longitudinal direction of the fastening rail are arranged on a base body 341 with a rectangular cross-section. Accordingly, a block 345 serving as abutment element has a passage 346 with a rectangular cross-section passing completely through this in the longitudinal direction. Two teeth 347 respectively project from the two wide inside walls of the passage 346 towards the opposite wall of the passage 346.

To clamp the band-shaped piece 118 in position, the fastening rail is inserted through the passage 346 until the four teeth 347 extend into four corresponding perforations 342 of the coupling section 340. As a result of a sloping side face of the teeth 347, displacement of the block 345 relative to the fastening rail is possible in only one direction.

A completely different locking mechanism is provided in the case of the coupling section 360 shown in FIGS. 29 to 31. While this fully corresponds to the coupling section 320 described in association with FIGS. 24 to 26, a block 365 has a different structure. In its base form it corresponds to the block 150 known from FIG. 2. This has a cylindrical hole 366 along its axis of symmetry, wherein one end of the block 365 is provided with a projection 367 projecting radially outwards in a ring shape. In addition, a locking projection 368 in a ring shape projecting radially towards the axis of symmetry is provided, said projection having two diametrically opposed breaks 369 and 370. The diameter of the hole 366 and the width of the coupling section 360 are coordinated to one another in such a way that the two toothed strips formed from equidistant teeth 362 on the narrow sides of the parallelepipedal base body 361 may be pushed through the hole 366 in such a way that the teeth 362 protrude into breaks 369 and 370. The fastening rail can thus be drawn through the hole 366 of the block 365 without friction and without locking in any form.

In order to fix a position of the block 365 relative to the fastening rail, the block 365 and the fastening rail are rotated relative to one another so that the locking projection 368 engages between two adjacent teeth 362. Thus, the position is doubly locked because of the two toothed rods. Such a locking position can be released again by rotating the block 365 back relative to the fastening rail by the teeth 362 being moved into the region of the breaks 369 and 370.

A further type of connection between a block 385 and a coupling section 380 is shown in FIGS. 32 and 33. The coupling section consists of rotationally symmetric spherical bodies 381 spaced from one another in a chain. The block 385 has a hollow cylindrical body section 382, which is completely sealed with a cover 383 of larger diameter. An elongated hole 384, one half of which facing away from the axis of symmetry is widened by a hole 386, is machined into the cover to be rotationally symmetric to its axis of symmetry.

A connection between the block 385 and the fastening rail is possible by directing the coupling section 380 through the hole 386 of the cover 383 of the block 385. To lock the block 385 with the fastening rail, the block 385 needs only be displaced relative to the fastening rail towards the axis of symmetry of the coupling section 380, and in fact in such a manner that the unwidened portion of the elongated hole 384 engages between two spherical bodies 381. In this position, the block 385 and the fastening rail are fixed relative to one another. This position may also be released by the fastening rail being displaced relative to the block 385 until the spherical bodies 381 can be directed through the hole 386 again.

A further possibility of connecting the fastening rail to a rotationally symmetric block 395 serving as abutment element is shown in FIGS. 34 and 35. The fastening rail is provided with a coupling section 390, which has an external thread 391. The block 395 comprises a central hole 396, which is provided with an internal thread 397 corresponding to the external thread 391. The block 395 has a hemispherical base body 398, on the arched side of which a cylinder section 399 is disposed. A parallelepipedal section 392, which can be brought into engagement with a fastening tool, e.g. a fork wrench, adjoins the flat side of the base body 398. A disc 393 with an opening 394 coordinated to the spherical surface of the base body 398 abuts against the spherical surface of the base body 398. As a result, the disc 393 can be rotated relative to the base body 398 and pivoted along the surface of the base body 398.

For insertion of the cruciate ligament replacement part 116, the fastening rail with the coupling section 390 is guided to the cylinder section 399 of the block 395 and the internal thread 397 screwed to the external thread 391. This enables an infinite adjustment of the prestress of the band-shaped piece 118 arranged on the free end of the fastening rail. The disc 393 allows adaptation to the surface of either the head 102 of the femur or the head 104 of the tibia in the case of an obliquely drilled longitudinal channel 106 or 108. The disc 393 always locates parallel to the surface thereof and because of the spherical configuration of the base body 393 can transfer the effective tensile forces onto the surface of the bones in an optimum manner.

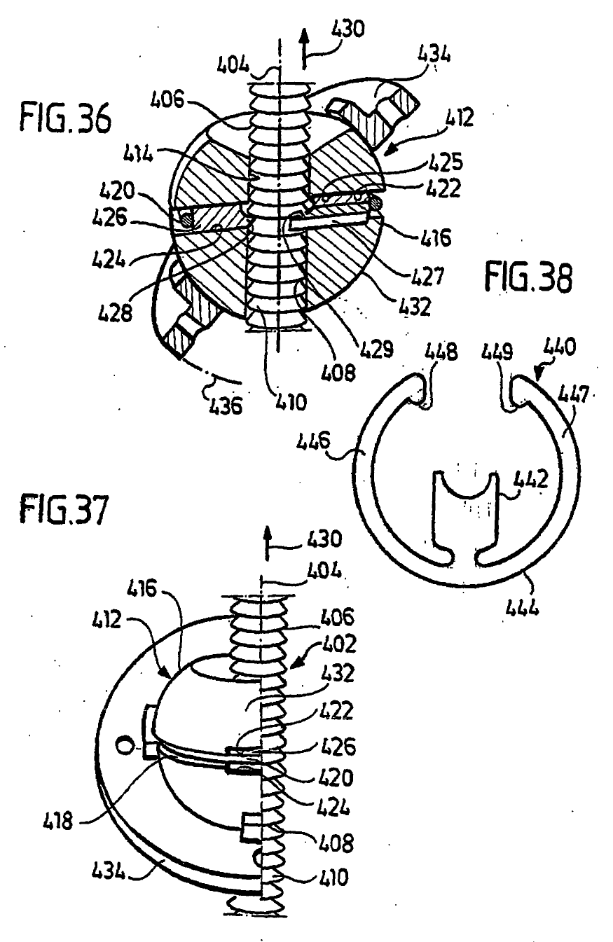

A ninth embodiment of a connecting element with associated abutment element is shown in FIGS. 36 and 37. The connecting element in the form of a fastening rail, only shown in sections, is provided with a coupling section 402, which essentially corresponds to the coupling section 260 of the first embodiment shown in FIG. 16. It is formed rotationally symmetric to a longitudinal axis 404 and has a plurality of identical annular recesses 406 so that an elongated structure results with a number of spherical bodies 410 with a cone base area 408 oriented transversely to the longitudinal axis 404 corresponding to the number of annular recesses 406, wherein the point of each cone body 410 extends into the cone base area 408 of the adjacent cone base body 410. As a result, the coupling section 402 has a Christmas tree-like appearance in side view.

As part of an abutment element 412 a sphere 416 having a central hole 414 is provided, wherein the inside diameter of the hole 414 is adapted to the largest outside diameter of the coupling section 402, so that the sphere is displaceable in the direction of the longitudinal axis 404 on the coupling section 402.

In an equatorial plane running transversely to the longitudinal axis 404, the sphere 416 has an annular groove 418 completely surrounding it, the depth and width thereof being adapted to a spring wire ring 20 disposed therein. A perforation 422 with a parallelepipedal cross-section oriented transversely to the longitudinal axis is provided in the equatorial plane, said perforation intersecting the hole 414, as a result of which two locking body receptacles 424 and 425 are formed, which respectively extend radially outwards from the hole 414 and into which a locking body 426 or 427 is respectively inserted. The two locking bodies 426 and 427 are held in the locking body receptacles 424 and 425 by the spring wire ring 420. An end 428 or 429 of the locking body 426 or 427 respectively facing the hole 414 is configured to correspond to the annular recesses 406 and can extend into these.

A ring-shaped disc 434 is disposed on the sphere surface 432 to enable rotation and pivoting movement so that any desired angle of inclination 436 between a plane defined by the disc 434 and the longitudinal axis 404 can be set.

For insertion of the cruciate ligament replacement part 116, the fastening rail with the coupling section 402 is pushed through the hole 414 in the direction of arrow 403 until the locking bodies 426 and 427 prestressed by the spring wire ring 420 towards the longitudinal axis 404 extend into the annular recesses 405 with their ends 428 and 429. Because of the configuration of the cone bodies 410, when the sphere 416 is held fast, the fastening rail with the coupling section 402 arranged thereon can only be moved in the direction of arrow 430, in the opposite direction the ends 428 and 429 located on the cone base area 408 act as a block for any movement. The band-shaped piece 118 arranged on the free end of the fastening rail can be prestressed as a result of corresponding relative movement between the fastening rail and the sphere 416. The disc 434 enables adaptation to the surface either of the head 102 of the femur or the head 104 of the tibia in the case of an obliquely drilled longitudinal channel 106 or 108. Because of the special mounting on the sphere 416, the disc 434 can always locate parallel to the surface of the head 102 of the femur or the head 104 of the tibia, as a result of which it can transfer the effective tensile forces onto the surface of the bones in an optimum manner because of the prestress of the band-shaped piece 118.

In place of the two locking bodies 426 and 427 described in association with FIGS. 36 and 37, a restraining element shown in FIG. 38 and given the overall reference 440 can be used. It comprises a holding element 442, which corresponds to the locking bodies 426 and 427 and is moulded symmetrically onto a C-shaped holding ring 444 made of a spring steel, so that the restraining element 440 has a flat configuration overall. As a result of the symmetrical arrangement of the holding element 442 on the holding ring 444, two spring arms 446 and 447 with dimensions selected so that they fit into the annular groove 418 are formed on the holding ring. Free ends of the spring arms 446 and 447 are provided with holding lugs 448 and 449, which point approximately onto a centre point of the holding ring 444. The length of the spring arms 446 and 447 is selected so that the holding lugs 448 and 449 extend at least partially into the parallelepipedal perforation 422 and hold the holding element 442 under prestress on the coupling section 402.

What is claimed is:

1. Implant for fastening of a tendon replacement part to a channel receiving the tendon replacement part in the region of the tibia and/or femur close to the knee, with an abutment element abutting against the channel and with a connecting element, wherein the tendon replacement part and the abutment element may be coupled by means of the connecting element, wherein the abutment element and the connecting element defining a longitudinal direction are configured and reconciled with to one another such that the tendon replacement part is tensingly fixable in the channel by relative movement between the abutment element and the connecting element in longitudinal direction.

2. Implant according to claim 1, wherein the connecting element and/or the abutment element are made from a body compatible material.

3. Implant according to claim 2, wherein the body compatible material is a resorbable plastic.

4. Implant according to claim 1, wherein the abutment element has at least one passage recess for the connecting element, so that the abutment element and the connecting element are movable relative to one another transversely to the longitudinal direction.

5. Implant according to claim 1, wherein the abutment element has at least one passage recess for the connecting element, so that the connecting element and the abutment element are displaceable relative to one another, and that abutment element has at least one fixture recess, wherein the at least one passage recess and the at least one fixture recess are connected to one another.

6. Implant according to claim 1, wherein the connecting element comprises a plurality of fixture recesses, which are arranged in the longitudinal direction of the connecting element.

7. Implant according to claim 5, wherein adjacent fixture recesses are spaced equidistant from one another.

8. Implant according to claim 5, wherein adjacent fixture recesses are separated from one another.

9. Implant according to claim 5, wherein the connecting element is provided with an engagement strip formed by fixture recesses.

10. Implant according to claim 9, wherein the connecting element has at least two engagement strips.

11. Implant according to claim 9, wherein a side face of the connecting element is provided with an engagement strip.

12. Implant according to claim 9, wherein an engagement strip is arranged on the connecting element between side faces of the connecting element.

13. Implant according to claim 9, wherein the engagement strip is configured in a sawtooth shape.

14. Implant according to claim 9, wherein an engagement strip comprises rectangular recesses as fixture recesses.

15. Implant according to claim 5, wherein the abutment element has a holding member, which is immersible into a fixture recess and by means of which the abutment element may be connected to the connecting element.

16. Implant according to claim 15, wherein a holding member comprises a plurality of holding teeth for simultaneous engagement into a plurality of fixture recesses.

17. Implant according to claim 15, wherein a holding member is arranged at an angle to an abutment face of the abutment element.

18. Implant according to claim 1, wherein the abutment element is connectable to the connecting element by a relative movement of the entire abutment element thereto.

19. Implant according to claim 5, wherein the abutment element is connectable to the connecting element by a relative movement of the entire abutment element thereto.

20. Implant according to claim 1, wherein the abutment element is connectable to the connecting element by relative movement of a holding part arranged on the abutment element thereto.

21. Implant according to claim 5, wherein the abutment element is connectable to the connecting element by relative movement of a holding part arranged on the abutment element thereto.

22. Implant according to claim 1, wherein the connecting element is a body that is inflexible in the longitudinal direction.

23. Implant according to claim 1, wherein the connecting element is dimensioned such that it has a larger width in a transverse direction to its longitudinal direction than in the transverse direction perpendicular thereto.

24. Implant according to claim 23, wherein the connecting element has an essentially rectangular cross-section at least in the region extending in the channel.

25. Implant according to claim 1, wherein the connecting element is configured to be essentially rotationally symmetric in its longitudinal direction.

26. Implant according to claim 25, wherein in the case of an essentially rotationally symmetric configuration of the connecting element, a fixture recess extends around the periphery of the connecting element.

27. Implant according to claim 25, wherein adjacent fixture recesses are separated by a spherical element.

28. Implant according to claim 21, wherein the holding part of the abutment element engages in the longitudinal direction into a fixture recess.

29. Implant according to claim 1, wherein the abutment element may be connected to the connecting element by rotation relative thereto.

30. Implant according to claim 5, wherein the abutment element may be connected to the connecting element by rotation relative thereto.

31. Implant according to claim 29, wherein the abutment element has a holding member, which is engageable into a fixture recess of the connecting element by rotation of the abutment element relative to the connecting element.

32. Implant according to claim 1, wherein the connecting element is flexible transversely to its longitudinal direction.

33. Implant according to claim 1, wherein the abutment element is fixable non-positively to the connecting element.

34. Implant according to claim 1, wherein the relative movement between the abutment element and the connecting element for fixing the abutment element to the connecting element is performed from a non-fixed position into a fixing position.

35. Implant according to claim 5, wherein the relative movement between the abutment element and the connecting element for fixing the abutment element to the connecting element is performed from a non-fixed position into a fixing position.

36. Implant according to claim 1, wherein the end of the connecting element facing the tendon replacement part has a tendon replacement part receptacle for passage or suspension of the tendon replacement part.

37. Implant according to claim 36, wherein the tendon replacement part receptacle is configured in a loop shape.

38. Implant according to claim 37, wherein the loop-shaped tendon replacement part receptacle has an essentially round loop shape.

39. Implant according to claim 37, wherein the loop-shaped tendon replacement part receptacle is essentially triangular.

40. Implant according to claim 36, wherein the tendon replacement part receptacle is configured in a hook shape.

41. Implant according to claim 40, wherein a locking element is provided on the free end of the hook-shaped tendon replacement part receptacle and at least one locking element receptacle is provided on the connecting element to move the hook-shaped tendon replacement part receptacle into a loop-shaped tendon replacement part receptacle and lock it in position.

42. Implant according to one of claim 36, wherein the cross-section of the tendon replacement part receptacle is round, oval or rectangular.

43. Implant according to claim 1, wherein the abutment element can be screwed to the connecting element.

44. Implant according to claim 43, wherein the passage recess of the abutment element is provided with an internal thread, and that the connecting element is provided with a corresponding external thread.

45. Implant according to claim 1, wherein the abutment element has a section extending into the channel.

46. Implant according to claim 45, wherein the extending section is configured to be rotationally symmetric.

47. Implant according to claim 45, wherein the extending section has a cross-section which decreases in the longitudinal direction of the passage recess in the direction towards the tendon replacement part.

48. Implant according to claim 1, wherein the abutment element has an abutment region abutting externally against the channel and at least partially covering the channel.

49. Implant according to claim 48, wherein the abutment region has an external face directed away from the channel, which runs essentially in a plane transversely to the longitudinal direction of the passage recess.

50. Implant according to claim 48, wherein the abutment region has an external face directed away from the channel, which with a plane transversely to the longitudinal direction of the passage recess encloses an angle, which corresponds to an angle formed between the channel and a passage face formed by the channel in the tibia or the femur.

51. Implant according to claim 48, wherein the abutment region has a larger cross-section than the immersing section.

52. Implant according to claim 48, wherein the abutment region is pivotally disposed on the immersing section.

53. Implant according to claim 15, wherein the holding member is displaceably disposed on the abutment element in a direction transverse to the passage recess.

54. Implant according to claim 53, wherein a guide recess running transversely to the passage recess is provided for guiding and supporting the holding member.

55. Implant according to claim 53, wherein the holding member is biased towards the passage recess.

56. Implant according to claim 55, wherein an elastic element is provided to generate a prestressing.

57. Implant according to claim 56, wherein the elastic element is configured in one piece with the holding member.

58. Implant according to claim 25, wherein the fixture recess comprises a fixture recess face oriented transversely, and one oriented obliquely, to the longitudinal direction of the connecting element.
